(19) United States
(12) Patent Application Publication
(10) Pub. No.: US 2014/0038132 A1
(43) Pub. Date: Feb. 6, 2014

(54) DENTAL REGENERATIVE DEVICE MADE OF POROUS METAL

(71) Applicant: Zimmer Trabecular Metal Technology, Inc., Parsippany, NJ (US)

(72) Inventors: Edward M. Willis, Hoboken, NJ (US); Ken Varner, Floral Park, NY (US); Michael Scott Collins, San Marcos, CA (US); Liis Underwood, San Marcos, CA (US)

(73) Assignee: Zimmer Trabecular Metal Technology, Inc., Parsippany, NJ (US)

(21) Appl. No.: 13/954,051

(22) Filed: Jul. 30, 2013

Related U.S. Application Data
(60) Provisional application No. 61/677,955, filed on Jul. 31, 2012.

(51) Int. Cl. A61C 8/02; A61K 6/04

(52) U.S. Cl. CPC A61C 8/0006; A61K 6/04
USPC 433/173

(57) ABSTRACT

A regenerative device can include a first side section, a second side section, and a top section extending between and connecting the first side section and the second side section. The top section and the first and second side sections can each be formed from a porous material that retains its structure after implantation in the patient. The regenerative device can be used for ridge augmentation of a maxilla or a mandible. The top section can include an opening configured for receiving an implant. The porous material can be permanently implanted in the mouth and promote bone regeneration or ridge augmentation. The first and second side sections of the device can include openings for receiving a fastener to secure the device to the alveolar ridge.

### CLAIM OF PRIORITY

This application claims the benefit of U.S. Provisional Patent Application Ser. No. 61/677,955, entitled “DENTAL REGENERATIVE DEVICE MADE OF POROUS METAL”, and filed on Jul. 31, 2012, the benefit of priority of which is claimed hereby, and which is incorporated by reference herein in its entirety.

### TECHNICAL FIELD

The present patent application relates to a dental procedure and more particularly, to an apparatus and method of regenerating bone of the alveolar ridge.

### BACKGROUND

Dental implants may be used for the replacement of all, or a portion of, a patient's teeth. Following the extraction of one or more teeth, the bone that forms the maxilla or the mandible may resorb in the area where the one or more teeth were removed. The resulting loss of bone may make it difficult to secure a dental implant in the patient's mouth where the one or more extracted teeth were previously positioned. As such, various materials may be used to restore the bone prior to, or as part of, a procedure to place the implant(s) inside the patient's mouth.

In some cases, a titanium mesh material can be used in combination with a grafting material, in order to help promote bone growth. In addition to or as an alternative to a metal material, a resorbable material can be used to regenerate bone.

### OVERVIEW

The present inventors have recognized, among other things, that there is an opportunity for a regenerative device and method that can maintain its structure and space in the mouth over time, while promoting bone growth. More particularly, the present inventors have recognized that a device formed from a porous material can retain its shape and structure after implantation in the mouth and can be used to regenerate bone of the alveolar ridge vertically and/or horizontally.

To better illustrate the dental regenerative device and methods disclosed herein, a non-limiting list of examples is provided here:

In Example 1, a regenerative device comprises a first side section, a second side section, and a top section extending between and connecting the first side section and the second side section. The top section and the first and second side sections can each be formed from a porous material that is configured to retain its structure after implantation in a patient. The top section can include an opening configured for receiving an implant.

In Example 2, the regenerative device of Example 1 is optionally configured such that the top section is curved such that the top section, the first side section, and the second side section form an elongated arch.

In Example 3, the regenerative device of any one of Examples 1 or 2 is optionally configured such that the top section is curved such that the regenerative device has a generally semi-circular shape.

In Example 4, the regenerative device of Example 1 is optionally configured such that the top section is planar.

In Example 5, the regenerative device of Example 4 is optionally configured such that the regenerative device has a pyramid shape.

In Example 6, the regenerative device of Example 4 is optionally configured such that the first side section and the second side section are parallel to one another.

In Example 7, the regenerative device of any one of Examples 1-6 is optionally configured such that the regenerative device is generally linear along an overall length of the regenerative device.

In Example 8, the regenerative device of any one of Examples 1-7 is optionally configured such that the regenerative device has a curvature along at least a portion of an overall length of the regenerative device such that the regenerative device is configured to conform with a shape of a maxilla or a mandible of the patient.

In Example 9, the regenerative device of any one of Examples 1-8 is optionally configured such that the first side section includes a first opening configured for receiving a first fastener to secure the device to an alveolar ridge of the patient, and the second side section comprises a second opening configured for receiving a second fastener to secure the device to the alveolar ridge.

In Example 10, the regenerative device of any one of Examples 1-9 is optionally configured such that the porous material is tantalum.

In Example 11, the regenerative device of any one of Examples 1-10 is optionally configured such that the top section comprises at least two openings.

In Example 12, the regenerative device of any one of Examples 1-10 is optionally configured such that the top section comprises at least three openings.

In Example 13, the regenerative device of any one of Examples 1-12 is optionally configured such that the opening has a generally rectangular shape.

In Example 14, the regenerative device of any one of Examples 1-12 is optionally configured such that the opening has a generally circular shape.

In Example 15, the regenerative device of any one of Examples 1-14 is optionally configured such that the regenerative device is configured for attachment to an alveolar ridge in a maxilla or a mandible of the patient and the opening in the top section is configured to receive a dental implant.

In Example 16, a ridge augmentation device comprises an elongated arch comprising a first side portion, a second side portion, and a top portion disposed between the first and second side portions. The elongated arch can be formed of a porous metal and configured to be secured to an alveolar ridge of a patient. The top portion can include an opening configured for receiving an implant.

In Example 17, the regenerative device of Example 16 is optionally configured such that the top portion includes at least two openings.

In Example 18, the regenerative device of any one of Example 16 or 17 is optionally configured such that the porous metal is tantalum.

In Example 19, the regenerative device of any one of Examples 16-18 is optionally configured such that the first side portion comprises an opening, configured for receiving a fastener to secure the arch to an alveolar ridge of the patient, and the second side portion comprises an opening, configured for receiving a fastener to secure the arch to the alveolar ridge.

In Example 20, a method of performing ridge augmentation of a maxilla or a mandible of a patient to regenerate bone includes exposing a portion of the bone that forms the alveolar ridge in the maxilla or the mandible of the patient by cutting through tissue covering the bone. The method further includes securing a regenerative device to the exposed portion of the bone, and closing the tissue around the exposed portion of the bone and the regenerative device. The regenerative device can include an opening configured for receiving an implant and can be formed of a porous metal that retains its structure after implantation in the maxilla or the mandible.

In Example 21, the method of Example 20 optionally further includes placing a membrane over the regenerative device, prior to closing the surrounding tissue around the exposed portion of the bone and the regenerative device.

In Example 22, the method of Example 21 is optionally configured such that the membrane is formed of a material that prevents soft tissue from growing into the porous metal of the regenerative device.

In Example 23, the method of any one of Examples 19-22 optionally further includes placing bone graft in and around the exposed bone, prior to securing the regenerative device to the bone.

In Example 24, the method of any one of Examples 19-23 is optionally configured such that securing the regenerative device to the exposed portion of the bone includes attaching a first side portion of the regenerative device to an alveolar ridge of the patient and attaching a second side portion of the regenerative device to the alveolar ridge.

In Example 25, the device or method of any one or any combination of Examples 1-24 is optionally configured such that all elements or options recited are available to use or select from.

These and other examples and features of the present regenerative devices, kits, and methods will be set forth in part in the following Detailed Description. This overview is intended to provide a summary of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of a mouth, including an area of the mandible where teeth have been removed.

FIG. 2 is a perspective view of the mouth of FIG. 1 after the tissue has been cut, exposing a portion of the bone forming the alveolar ridge of the mandible.

FIG. 3 is a perspective view of an example of a dental regenerative device in accordance with the present application.

FIG. 3A is an end view of the dental regenerative device of FIG. 3.

FIG. 3B is a side view of a first side of the dental regenerative device of FIG. 3.

FIGS. 4-9 are perspective views of examples of dental regenerative devices in accordance with the present application.

### DETAILED DESCRIPTION

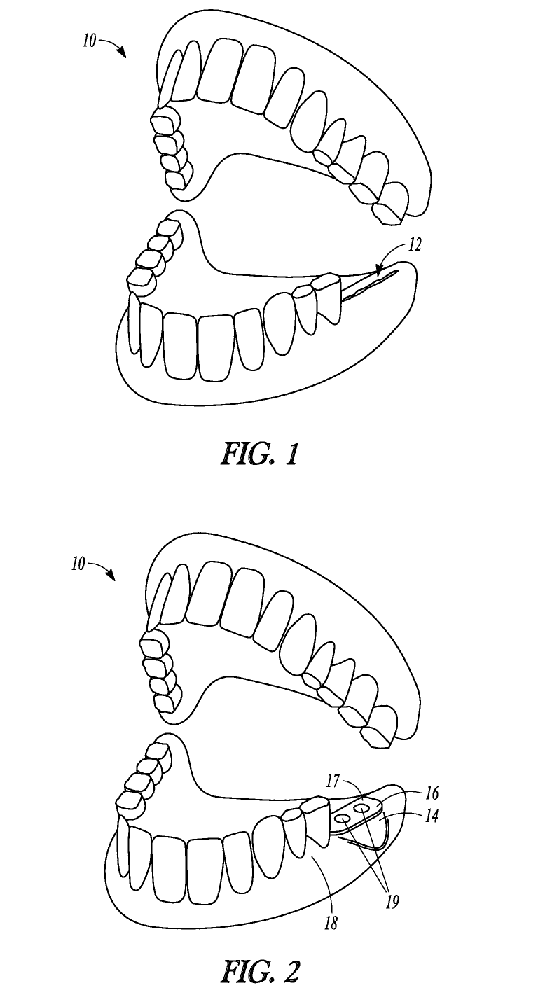

FIG. 1 shows an inside of a patient's mouth 10 after the removal of one or more teeth from an area 12 in the mouth 10. The teeth may have been removed, for example, due to extensive decay or other damage.

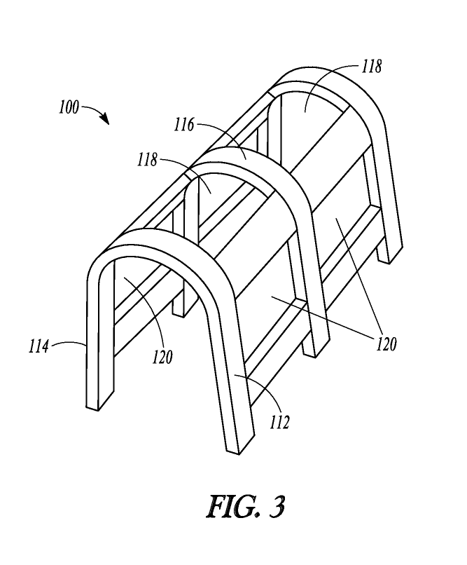

FIG. 2 shows the mouth 10 after a portion of tissue 14 has been cut and then pulled back, exposing a portion of bone 16 that forms an alveolar ridge 17 in the mouth 10. More specifically, the bone 16 is part of the alveolar ridge 17 that forms a mandible 18, or lower jaw, of the mouth 10. Holes 19 are shown in FIG. 2 where the one or more removed teeth previously resided. Particularly, FIG. 2 depicts two holes 19 that correspond with two teeth that have been removed from the mandible 18 of the mouth 10. However, more or less teeth can be removed from the mandible 18 in a similar procedure. Furthermore, one or more teeth can similarly be removed from the maxilla 22, or upper jaw, of the mouth 10.

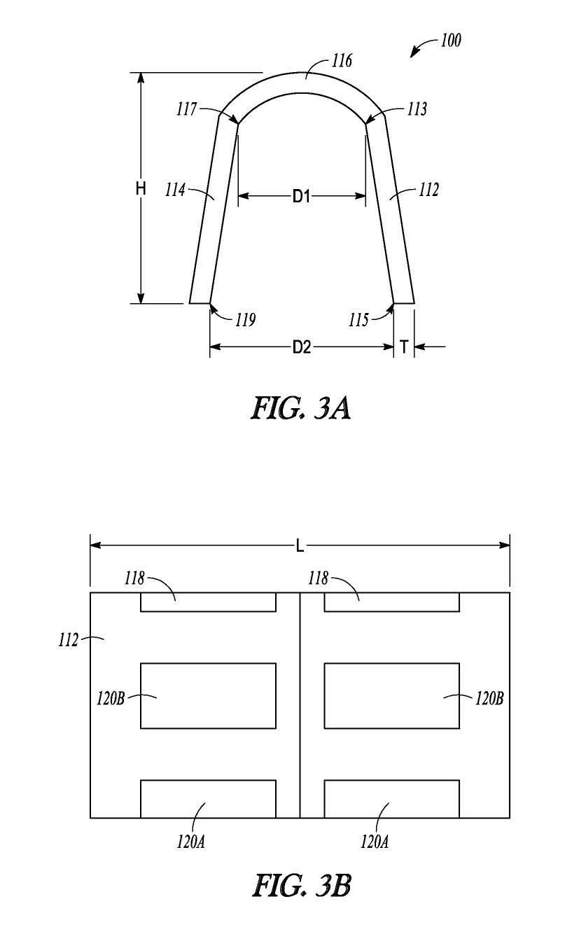

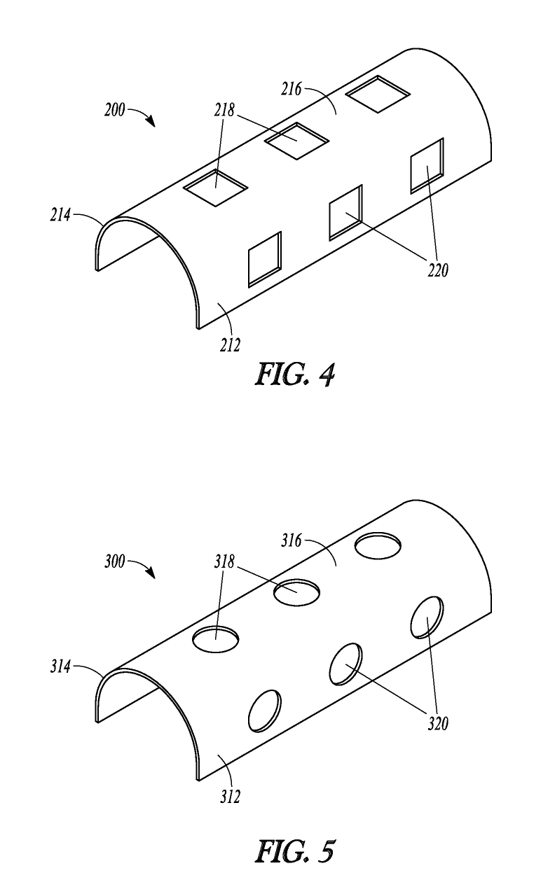

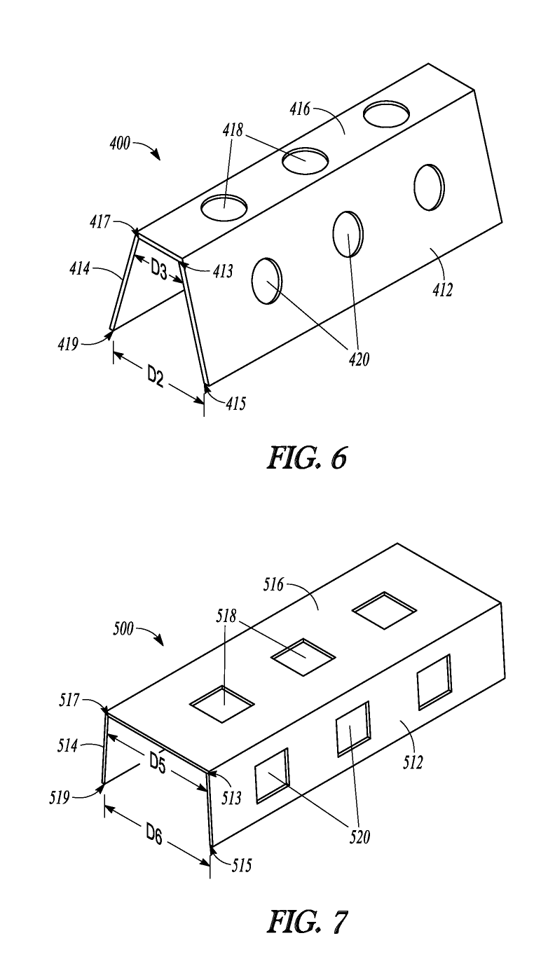

FIG. 3 shows an example of a dental regenerative device 100 that can be implanted inside the mouth 10 in an area, similar to the area 12 shown in FIG. 1, where one or more teeth have been removed. Once the one or more teeth have been removed from the mouth 10, bone absorption can occur in the surrounding area, and in some cases, such bone absorption can occur rapidly. The dental regenerative device 100 can be used to facilitate regrowth of the bone 16 in the area 12. For example, the regenerative device 100 can be used for vertical bone growth (to regain or increase a height of the bone 16) and/or horizontal bone growth. This bone growth or bone regrowth can also be described using alternative terms, such as, for example, ridge augmentation or bone augmentation.

The dental regenerative device 100 can be formed from at least one material that can allow the dental regenerative device 100 to maintain a desired shape inside the mouth 10. In an example, the dental regenerative device 100 can be formed from a biocompatible, rigid, porous material that is able to retain its shape and structure long after implantation in the mouth 10. Because the device 100 can be formed of a rigid material, the device 100 can be implanted inside the mouth 10 without a supplemental support structure. However, in examples, the device 100 can be paired with a supplemental support structure if desired. This is in contrast to other designs, which require a support material to be used in combination with the regenerative material, in which case, the support material may need to be removed from the mouth in a subsequent surgery.

In an example, the dental regenerative device 100 can be formed of a porous structure, such as to facilitate bone ingrowth or regrowth. A porous biomaterial can be useful as a bone substitute, and can have a porosity as low as 55%, 65%, or 75%, or as high as 80%, 85%, or 90%, or within any range defined by any of the foregoing values. In an example, the porous structure can include or can be formed of a material produced using Trabecular Metal™ Technology, generally available from Zimmer, Inc. of Warsaw, Ind. Trabecular Metal™ is a trademark of Zimmer, Inc. Such a material can be formed using a foamed polymer (such as polyurethane, as one example) that can be reduced to a reticulated vitreous carbon foam substrate or skeleton. The carbon skeleton can be infiltrated and coated with a first layer of biocompatible metal, such as tantalum, to produce a low density material, and then plated with a second layer of tantalum to produce a high density material. The metal can be deposited on the carbon substrate by a chemical vapor deposition (CVD) process, such as in the manner disclosed in detail in U.S. Pat. No. 5,282,861, the disclosure of which is incorporated herein by reference. One or more other metals, e.g., in addition to tantalum, including alloys thereof, can be used, such as, for example, niobium.

Generally, the porous structure can include a large plurality of ligaments defining open spaces there between, with each ligament generally including a carbon core covered by a thin film of metal, such as tantalum, for example. The open spaces between the ligaments can form a matrix of continuous channels, such as having no dead ends, such as to permit uninhibited growth of cancellous bone through the porous tantalum structure. The porous structure can include up to 75%-85% or more void space therein. In an example, a porous tantalum structure can provide a lightweight, strong porous structure that can be substantially uniform and consistent in composition, and that can closely resemble the structure of natural cancellous bone, which can thereby provide a matrix into which cancellous bone can grow. The porous tantalum structure can be made in a density selected from a variety of densities, such as to selectively tailor the structure for a particular application. The porous tantalum can be fabricated to permit selecting virtually any desired porosity and pore size, and can thus be matched with the surrounding natural bone, such as to provide an improved matrix for bone ingrowth and mineralization.

Because the dental regenerative device 100 can be formed of a porous material, bone can grow into and through the device 100. The device 100 can remain inside the mouth of the patient for a long period or indefinitely.

In an example, the dental regenerative device 100 can be used with a membrane. After the dental regenerative device 100 is implanted inside the mouth, the membrane can be placed over the dental regenerative device 100 during the same surgery or during a subsequent surgery. The membrane can be used as a barrier to prevent soft tissue growth through the dental regenerative device 100 in order to enable bone growth through the device 100. The membrane can be formed of any material(s) that can act as a soft tissue barrier, and such materials can include, for example, collagen and pericardium. The membrane can be a temporary membrane that can be removed from the body in a later surgery, or the membrane can be formed from a resorbable material, such as collagen.

The dental regenerative device 100 can include a first side section 112, a second side section 114, and a top section 116 disposed between the first side section 112 and the second side section 114. The dental regenerative device 100 can be a continuous piece which can include the first 112 and second 114 side sections and the top section 116. At least a portion of the first 112 and second 114 side sections can be generally parallel to one another, or the first 112 and second 114 side sections can extend in non-parallel planes. The top section 116 can be curved, as shown in FIG. 3, such that the top section 116 and the first 112 and second 114 side sections form an elongated arch.

The top section 116 can include one or more windows or openings 118 configured to receive an implant, as described below. Thus, although FIG. 3 depicts two openings 118 in the top section 116, more or less than the two openings 118 can be provided. The openings 118 can be generally rectangular in shape, as shown in FIG. 3, or the openings 118 can have a different shape, such as, for example, a circular shape or an irregular shape. The size of the openings 118 can also be larger or smaller than the size of the openings shown in FIG. 3. In addition to or as an alternative to receiving an implant(s), the openings 118 can be configured to reduce or minimize an overall weight of the dental regenerative device 100, as well as the total amount of material used to form the dental regenerative device 100.

The openings 118 can be structured to receive an implant, which can typically include a screw or a shaft that can be placed into a bore formed in the bone 16 and used to support a dental prosthesis. Common materials for dental implants can include, for example, titanium or an alloy thereof. A first portion of the implant can be configured for implantation within the bone 16 and a second portion of the implant can be configured to extend above the gum line of the mouth 10. The second portion of the implant can engage with or receive a dental prosthesis, such as, for example, a crown or a bridge. When placing the dental regenerative device 100 inside the mouth, the openings 118 on the top section 116 can thus be aligned with the desired implant locations inside the mouth 10.

Because there are two openings 118, as shown in FIG. 3, at least two implants can be used with the dental regenerative device 100, with one implant placed inside each opening 118. Alternatively, because each of the openings 118 occupies a significant amount of the top section 116 in the example shown in FIG. 3, it can be possible to place two or more implants in each of openings 118, depending, at least in part, on a size of the openings 118 relative to the size of the implants. As discussed further below, the size and shape of the openings 118 can vary.

The implant(s) can be ‘loaded’ or placed inside the mouth 10 at the same time that the dental regenerative device 100 is implanted inside the mouth. The excess space around the implant and the dental regenerative device 100 can be packed with bone graft and/or bone filler. Alternatively, the implant(s) can be loaded as a part of a separate surgery that takes place at a later date following implantation of the dental regenerative device 100, such as to give the bone 16 time to regenerate around and through the dental regenerative device 100 before loading the implant(s). For example, loading of the implant at a later date can allow the bone 16 time to regenerate vertically through the use of the dental regenerative device 100.

As shown in FIG. 3, each of the first 112 and second 114 side sections can include windows or openings 120, which are shown in FIG. 3B and described in further detail below.

FIG. 3A shows an end of the dental regenerative device 100, including the first 112 and second 114 side sections, and the top section 116. The first side section 112 can include a top edge 113 and a bottom edge 115, while the second side section 114 can include a top edge 117 and a bottom edge 119. As shown in FIG. 3A, the first 112 and second 114 side sections can extend outward relative to the top section 116, in a non-parallel relationship, such that a distance D1 between the top edges 113 and 117 of the first 112 and second 114 side sections, respectively, can be less than a distance D2 between the bottom edges 115 and 119 of the first 112 and second 114 side sections, respectively. In an example, the first 112 and second 114 side sections can extend inward relative to the top section 116, in a non-parallel relationship, such that the first distance D1 can be greater than the second distance D2. In an example, the first 112 and second 114 side sections can be essentially parallel, such that the first distance D1 is substantially equivalent to the second distance D2.

The dental regenerative device 100 can be available in a variety of sizes, as well as shapes (described below), based on patient anatomy, including, for example, the patient's jaw line and the number of teeth that have been removed in an area of the mouth 10 intended to receive the dental regenerative device 100. The dental regenerative device 100 can include an overall height H and a thickness T, both of which can vary. The range of values of the height H can be based, at least in part, on an average height of the alveolar ridge 17 in humans.

The thickness T of the dental regenerative device 100 can be thick enough that the device 100 has structural integrity and thin enough to use a minimal amount of material and/or permit the device to bend or flex by at least a minimal amount. In an example, the thickness T of the dental regenerative device can be from about 0.01 inches (about 0.25 millimeters) to about 0.2 inches (about 5.1 millimeters). In another example, the thickness T can be from about 0.02 inches (about 0.51 millimeters) to about 0.08 inches (about 2.0 millimeters). In another example, the thickness T can be about 0.025 inches (about 0.64 millimeters). In another example, the thickness T can be about 0.05 inches (about 1.27 millimeters). The thickness T of the first 112 and second 114 side sections and the top section 116 can be substantially equivalent. In an example, a thickness of the top section 116 can be different than a thickness of at least one of the first 112 and second 114 side sections. In an example, a thickness can vary within each section; for example, the first side section 112 can be thinner near the top edge 113 as compared to the bottom edge 115.

FIG. 3B is a side view of the first side section 112. The first 112 and second 114 side sections can be generally identical in configuration. In an example, the first 112 and second 114 side sections can have different configurations, such as, for example, a different number or location of the openings 120. The dental regenerative device 100 can include an overall length L, and as described above, the dental regenerative device 100 can be available in different sizes having different lengths L. In an example, the dental regenerative device 100 can be generally linear along the overall length L. The dental regenerative device 100 can also have a curvature along the overall length L.

FIG. 3B shows four openings 120 in the first side section 112, as well as a portion of the openings 118 located primarily in the top section 116 and described above. Similar to the openings 118 in the top section 116, the openings 120 in the first 112 and second 114 side sections can be used to minimize the overall weight and material of the dental regenerative device 100. Alternatively or additionally, the openings 120 can be configured for securing or fixing the dental regenerative device 100 to the alveolar ridge 17, as described below.

Two openings 120 can be located generally at a bottom portion of the first side section 112 and are labeled as openings 120A. The openings 120A can be used to minimize an overall amount of material used to form the dental regenerative device 100. Two openings 120 can be located generally in a mid-section of the first side section 112 and are labeled as openings 120B. The openings 120B can be used to minimize the overall amount of material of the device 100 and/or to aid in securing the device 100 inside the mouth.

The dental regenerative device 100 can be secured to the alveolar ridge 17 using known methods and fasteners for fixing a dental implant inside the mouth 10. Such fasteners can include, for example, tacks and screws, including bone screws. In an example, bone screws, which are commonly made of titanium or an alloy thereof, can be used to fix either or both of the first 112 and second 114 side sections to the alveolar ridge 17. In an example, the two openings 120B can be configured to receive two fasteners for securing the first side 112 to the alveolar ridge 17. Similarly, two openings 120 on the second side section 114 can be configured to receive two fasteners for securing the second side 114 to the alveolar ridge 17. Depending on a size of the openings 120B and a size of the fasteners, in an example, each of openings 120B can be configured to receive more than one fastener, such as, for example, a fastener in each end of each opening 120B.

The number of the openings 120B and the number of fasteners used to secure the dental regenerative device 100 can vary, and can depend, at least in part, on an overall size of the device 100 and where in the mouth 10 the device 100 is being implanted. The number of fasteners used can be based, at least in part, on ensuring that the device 100 is stable inside the mouth 10 once it is implanted and that the device 100 is restricted beyond minimal movement.

As shown in FIG. 3B, the openings 120B, as well as the openings 120A, can be generally rectangular in shape. In an example, the openings 120A and/or the openings 120B can have different shapes, such as, for example, a square or circular shape.

The openings 118 and 120 can be preformed in the top section 116 and the first 112 and second 114 side sections, respectively. In an example, the openings 118 and 120 can be formed (for example, drilled out) after the dental regenerative device 100 is made and prior to, or during, surgery to implant the dental regenerative device 100. Whether any or all of the openings 118 and 120 are preformed can depend on a size of the openings 118 and 120. It can be advantageous to form larger-sized openings when the dental regenerative device 100 is made to avoid having to later remove, for example, by drilling, a significant amount of material to form larger-sized openings. In some cases, it can be desirable to limit any alterations to the dental regenerative device 100 once the surgery has been started, especially once the device 100 has been placed in the mouth. As such, it can be desirable to determine a location of the openings 118 and 120 prior to implantation of the dental regenerative device 100.

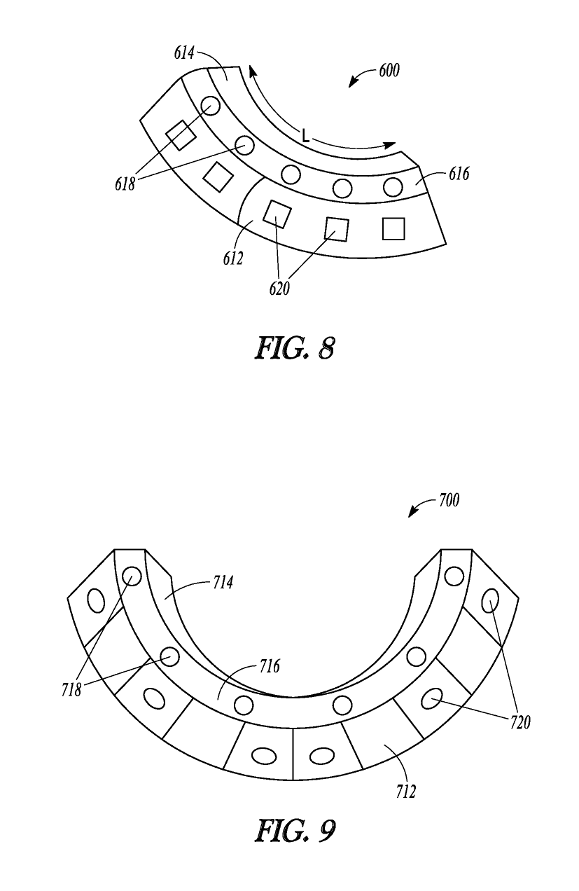

The dental regenerative device 100 shown in FIGS. 3, 3A and 3B can be structured as an elongated arch. However, numerous other shapes and sizes for the dental regenerative devices in accordance with the present patent application are also possible. FIG. 4 shows an example of a dental regenerative device 200, which similar to the dental regenerative device 100, can include a first side section 212, a second side section 214, and a top section 216. The first 212 and second 214 side sections and the top section 216 can be a continuous piece that can form an elongated arch. As compared to dental regenerative device 100, the first 212 and second 214 side sections of the dental regenerative device 200 can have a more round or circular shape.

The top section 216 can include openings 218 which can be configured for receiving implants as described above. As shown in FIG. 4, the openings 218 can be generally rectangular in shape. Three openings 218 can be included in the top section 216, such that the dental regenerative device 200 can receive three implants. More or less openings 218 can be included in an example of the dental regenerative device 200.

The first 212 and second 214 side sections can include openings 220. As described above in reference to the dental regenerative device 100, the openings 200 can be configured for receiving a fastener or other fixation device to secure the device 200 inside the mouth. Similar to the openings 218, the openings 220 can be generally rectangular in shape. More or less openings 220 can be included in an example of the dental regenerative device 200.

FIG. 5 shows an example of a dental regenerative device 300, which can include a first side section 312, a second side section 314, and a top section 316. Similar to the dental regenerative device 200, the dental regenerative device 300 can have a generally circular or semi-circular shape. The top section 316 can include openings 318 that can be generally circular in shape. Similarly, openings 320 can be generally circular or semi-circular in shape.

FIG. 6 shows an example of a dental regenerative device 400, which can include a first side section 412, a second side section 414, and a top section 416. The top section 416 can be generally flat or planar such that the top section 416 forms a shelf-like surface between the first 412 and second 414 side sections. The first side section 412 can include a top edge 413 and a bottom edge 415. The second side section 414 can include a top edge 417 and a bottom edge 419. A distance D3 between the top edges 413 and 417 of the first 412 and second 414 side sections, respectively, can be less than a distance D4 between the bottom edges 415 and 419 of the first 412 and second 414 side sections, respectively, such that the dental regenerative device 400 can have a generally pyramid shape. Comparing FIG. 6 to FIG. 3A, a difference between the distances D3 and D4 of the dental regenerative device 400 can be more pronounced or greater than a difference between the distances D1 and D2 of the dental regenerative device 100. The top section 416 can include three generally circular openings 418. The first 412 and second 414 side sections can include three generally circular openings 420.

FIG. 7 shows an example of a dental regenerative device 500, which can include a first side section 512, a second side section 514, and a top section 516. The top section 516 can be generally flat or planar, similar to the dental regenerative device 400 of FIG. 6. The first side section 512 can include a top edge 513 and a bottom edge 515. The second side section 514 can include a top edge 517 and a bottom edge 519. The first 512 and second 514 side sections can be essentially parallel to one another; as such, a distance D5 between the top edges 513 and 517 of the first 512 and second 514 side sections, respectively, can be generally equal to a distance D6 between the bottom edges 515 and 519 of the first 512 and second 514 side sections, respectively. As shown in FIG. 7, the dental regenerative device 500 can have a generally rectangular shape. The top section 516 can include three openings 518, generally rectangular in shape. The first 512 and second 514 side sections can include three openings 520, generally rectangular in shape.

Various configurations of the dental regenerative device are described above and shown in FIGS. 3-7. The dental regenerative device of the present application can include any combination of the various features described above in any of the example dental regenerative devices, including an overall size, overall shape, number of openings, size of openings, and shape of openings. For example, the dental regenerative device 200 of FIG. 4 can have generally circular openings instead of the rectangular openings shown in FIG. 4 on the top section 216 and/or the first 212 and second 214 side sections. Moreover, the dental regenerative device 200 can have a longer overall length or a shorter overall length to receive more or less implants.

In examples described herein, the first and second side sections of the dental regenerative device can be generally identical in size and configuration. The first and second side sections can be different; for example, a dental regenerative device can have a first side section with more openings and/or larger openings, as compared to the second side section.

In the examples described above and shown in FIGS. 3-7, the dental regenerative device can be generally straight or linear relative to an overall length of the device. FIG. 8 shows an example of a dental regenerative device 600 that can have a curve or a bend such that the dental regenerative device forms a shape configured to conform with a non-linear shape of the maxilla or the mandible of the patient. The dental regenerative device 600 can have a greater overall length L, as compared to, for example, the dental regenerative devices 100 and 200, such that the dental regenerative device 600 can be used in an area of the mouth, for example, where more than 2, 3 or even four teeth can be removed. Because the jaw line of both the maxilla and the mandible has a generally circular or semi-circular shape, a non-linear design of the dental regenerative device 600 can conform to such shape of the jaw line. The dental regenerative device 600 can include a curvature or an arc along at least a portion of its overall length L such that the dental regenerative device 600 can be configured to conform with a shape of the maxilla or the mandible of the patient. The dental regenerative device 600 can include a first 612 and a second 614 side section, and a top section 616. Each of the first 612 and second 614 side sections and the top section 616 can include openings 620 and 618, respectively. As similarly described above, a size and a shape of the openings 618 and 620 can vary.

FIG. 9 shows an example of a dental regenerative device 700 that can also include an arc or curvature along an overall length of the device 700, such that the device 700 can have a generally semi-circular shape. Similar to the dental regenerative device 600, the dental regenerative device 700 can be used when several teeth have been removed. In an example, the dental regenerative device 700 can be used when all or nearly all of the teeth have been removed from the mandible or maxilla of a patient. The dental regenerative device 700 can include a first 712 and a second 714 side section, and a top section 716. Each of the first 712 and second 714 side sections and the top section 716 can include openings 720 and 718, respectively, which can vary in size and shape.

As described above, the dental regenerative device 100 (see FIG. 3) can be formed of a porous structure, such as, for example, a porous tantalum structure. Any or all of the dental regenerative devices 200, 300, 400, 500, 600 and 700 can also be formed of a porous structure described above.

In an example, a patient-specific dental regenerative device can be prepared for a specific patient, prior to surgery, such as based on a size and shape of the patient's alveolar ridge in an area of the maxilla or mandible where teeth have been removed. The user can design the dental regenerative device with the number of openings, as well as the size of the openings, suited for the particular patient. As mentioned above, the user may want to minimize an amount of material used to form the dental regenerative device, while maintaining structural integrity of the device and facilitating fixation of implants and attachment mechanisms.

A set of dental regenerative devices can be provided in a kit to allow the user to select a dental regenerative device from a variety of devices having different sizes, shapes, etc. The kit can include dental regenerative devices having preformed openings and dental regenerative devices that can have some or all of the openings formed intra-operatively. The kit can provide the user flexibility, if, for example, the user initially chooses a particular dental regenerative device that is not an ideal size and/or shape for a particular patient's mouth. The user can then select another dental regenerative device from the kit. In an example, the kit can be pre-packaged and can include a hermetic seal. The components inside the kit can be sterilized prior to packaging or the kit can be formed of a material capable of withstanding sterilization. In an example, the kit can include instructions for use (IFUs) for selecting a dental regenerative device for a particular application. The instructions for use can be included inside the kit or accompany the kit.

The dental regenerative device is described herein as being used to promote vertical and/or horizontal bone growth in the mouth, such that dental implants can be loaded into the mouth. In some cases, a patient's bone loss or bone resorption can be significant enough that, even with bone regeneration or ridge augmentation, implants may not be suitable, and the patient may have to use dentures instead. The dental regenerative device described herein can be used to regenerate a sufficient amount of bone such that dentures can be better placed inside the patient's mouth.

The present disclosure includes a method of performing ridge augmentation of a maxilla or a mandible of a patient in order to regenerate bone. The method can include making an incision or cut in the tissue in an area where a dental regenerative device is intended to be implanted. The teeth typically have been previously removed from the intended area. The tissue can be pulled back, exposing the bone. Bone grafting material and/or bone filler can be used depending, at least in part, on the extent of bone resorption in the area. The method can include securing the dental regenerative device to the exposed portion of the bone. A first and/or a second side of the dental regenerative device can be attached to the alveolar ridge using known fixation devices and methods. As described above, the openings for receiving the fixation devices can be included in the dental regenerative device, as made, or the openings can be formed prior to or as part of the implantation surgery. In an example, a membrane can be placed over the regenerative device, such as to prevent soft tissue growth through the dental regenerative device before bone growth can occur. After the dental regenerative device and, if applicable, the membrane, are secured inside the mouth, the tissue around the bone can be closed. As mentioned above, the implants can be loaded during this same surgery or in a later surgery, such that the bone has time to regenerate with the aid of the dental regenerative device.

The dental regenerative device described herein can have versatility and flexibility for use in different areas of the mouth. The dental regenerative device can include a variety of shapes and sizes, and can be used with known fixation methods and devices to secure the device inside the mouth. Because the device can be formed from a porous metal material, suitable for permanent implantation in the mouth, the device does not have to be removed from the mouth in a later surgery. Because the porous metal material can form a rigid structure, the device can be used without an accompanying support structure that may require a later surgery to remove the support structure. The porous metal material can facilitate vertical bone growth and horizontal bone growth around the area in the mouth where the device is implanted, such that the device helps in regenerating the bone in the alveolar ridge prior to and/or after the dental implants are loaded inside the mouth.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A regenerative device comprising:
a first side section;
a second side section;
a top section extending between and connecting the first side section and the second side section, the top section and the first and second side sections each being formed from a porous material that is configured to retain its structure after implantation in a patient; and
an opening formed in the top section, the opening configured for receiving an implant.

2. The regenerative device of claim 1, wherein the top section is curved such that the top section, the first side section, and the second side section form an elongated arch.

3. The regenerative device of claim 1, wherein the top section is curved such that the regenerative device has a generally semi-circular shape.

4. The regenerative device of claim 1, wherein the top section is planar.

5. The regenerative device of claim 4, wherein the regenerative device has a pyramid shape.

6. The regenerative device of claim 4, wherein the first side section and the second side section are parallel to one another.

7. The regenerative device of claim 1, wherein the regenerative device is generally linear along an overall length of the regenerative device.

8. The regenerative device of claim 1, wherein the regenerative device has a curvature along at least a portion of an overall length of the regenerative device such that the regenerative device is configured to conform with a shape of a maxilla or a mandible of the patient.

9. The regenerative device of claim 1, wherein the first side section includes a first opening configured for receiving a first fastener to secure the device to an alveolar ridge of the patient, and the second side section comprises a second opening configured for receiving a second fastener to secure the device to the alveolar ridge.

10. The regenerative device of claim 1, wherein the top section comprises at least two openings.

11. The regenerative device of claim 1, wherein the opening has a generally rectangular shape.

12. The regenerative device of claim 1, wherein the opening has a generally circular shape.

13. The regenerative device of claim 1, wherein the regenerative device is configured for attachment to an alveolar ridge in a maxilla or a mandible of the patient and the opening in the top section is configured to receive a dental implant.

14. A ridge augmentation device comprising:
an elongated arch comprising a first side portion, a second side portion, and a top portion disposed between the first and second side portions, wherein the elongated arch is formed of a porous metal and configured to be secured to an alveolar ridge of a patient, the top portion including an opening configured for receiving an implant.

15. The ridge augmentation device of claim 14, wherein the top portion includes at least two openings.

16. The ridge augmentation device of claim 14, wherein the porous metal is tantalum.

17. The ridge augmentation device of claim 14, wherein the first side portion comprises an opening, configured for receiving a fastener to secure the arch to an alveolar ridge of the patient, and the second side portion comprises an opening, configured for receiving a fastener to secure the arch to the alveolar ridge.

18. A method of performing ridge augmentation of a maxilla or a mandible of a patient to regenerate bone, the method comprising:
exposing a portion of the bone that forms the alveolar ridge in the maxilla or the mandible of the patient by cutting through tissue covering the bone;
securing a regenerative device to the exposed portion of the bone, the regenerative device including an opening configured for receiving an implant and formed of a porous metal that retains its structure after implantation in the maxilla or the mandible; and
closing the tissue around the exposed portion of the bone and the regenerative device.

19. The method of claim 18, further comprising:
placing a membrane over the regenerative device, prior to closing the surrounding tissue around the exposed portion of the bone and the regenerative device.

20. The method of claim 19, wherein the membrane is formed of a material that prevents soft tissue from growing into the porous metal of the regenerative device.

21. The method of claim 18, further comprising:
placing bone graft in and around the exposed bone, prior to securing the regenerative device to the bone.

22. The method of claim 18, wherein securing the regenerative device to the exposed portion of the bone includes attaching a first side portion of the regenerative device to an alveolar ridge of the patient and attaching a second side portion of the regenerative device to the alveolar ridge.
